(19) United States
(12) Patent Application Publication
(10) Pub. No.: US 2021/0402057 A1
(43) Pub. Date: Dec. 30, 2021

(54) PROCESS FOR OBTAINING DECELLULARIZED EXTRACELLULAR MATRIX, DECELLULARIZED EXTRACELLULAR MATRIX, USE THEREOF AND KIT

(71) Applicants: Edmar Maciel Lima JÚNIOR, Fortaleza (BR); Universidade Federal do Ceará, Fortaleza (BR)

(72) Inventors: Manoel Odorico De Moraes FILHO, Fortaleza (BR); Maria Elisabete Amaral De MORAES, Fortaleza (BR); Felipe Augusto Rocha RODRIGUES, Fortaleza (BR); Carlos Roberto Koscky PAIER, Fortaleza (BR)

(21) Appl. No.: 17/294,305

(22) PCT Filed: Nov. 14, 2019
(86) PCT No.: PCT/BR2019/050495

§ 371 Date: May 14, 2021

(30) Foreign Application Priority Data
Nov. 14, 2018 (BR) 102018073515-2

(51) Int. Cl. A61L 27/36; A61K 35/60

(52) U.S. Cl. CPC A61L 27/362; A61L 27/3687; A61K 35/60

(57) ABSTRACT

The present invention describes a process for obtaining extracellular matrix from the skin of tilapia (Oreochromis niloticus) comprising the steps of chemical and enzymatic decellularization, detoxification, chemical disinfection, crosslinking, bleaching, dehydration and sterilization by gamma radiation, more specifically the steps comprised by each of said procedures and use of the extracellular matrix for treating ruptured of tissues, dermatitises, acute, chronic and traumatic lesions, battlefield wounds, necrotic wounds, lacerations, abrasions, bruises and other lesions and conditions. The present invention falls within the fields of pharmacy, medicine and veterinary medicine, dentistry, chemistry, tissue engineering, molecular biology and biotechnology.

### FIELD OF THE INVENTION

The present invention describes a process for obtaining an extracellular matrix from the skin of tilapia (Oreochromis niloticus) comprising the steps of chemical and enzymatic decellularization, detoxification, disinfection, crosslinking, bleaching, dehydration, and sterilization by gamma radiation. The present invention falls within the fields of pharmacy, medicine, veterinary medicine, chemistry, dentistry, tissue engineering, molecular biology and biotechnology.

### BACKGROUND OF THE INVENTION

Currently, in Brazil, there is no alternative of animal origin extracellular matrix developed in the country, which can be incorporated to the receptor organism, without the need for changes or removal. In developed countries, particularly in the United States, industrialized extracellular matrixes have been used with this purpose and in large scale, for several decades. The importation of these products to Brazil has been contributing even more to burden the Brazilian commercial balance, considering the elevated cost thereof and the economic reality of the country.

In the search for the state of the art in scientific and patent literature, the following documents were found, which deal with the theme:

Document CN108355172 differs from the present invention as it presents the process for obtaining different decellularized fish skin.

Munnelly, Amy E., et al. “Porcine vena cava as an alternative to bovine pericardium in bioprosthetic percutaneous heart valves.” Biomaterials 33.1 (2012): 1-8.

Badylak, Stephen F. “The extracellular matrix as a scaffold for tissue reconstruction Seminars in cell & developmental biology. Vol 13. No. 5. Academic Press, 2002.

Badylak, Stephen F., Donald O. Freytes, and Thomas W. Gilbert. “Extracellular matrix as a biological scaffold material: structure and function.” Acta biomaterialia 5.1 (2009): 1-13.

Flwang, Julia, et al. “Scaffold for connective tissue repair.” U.S. Pat. No. 8,226,715. 24 Jul. 2012.

Witt, Joana, et al. “Decellularised conjunctiva for ocular surface reconstruction.” Acta biomaterialia 67 (2018): 259-269.

Vastine, David W., William B. Stewart, and Ivan R. Schwab. “Reconstruction of the periocular mucous membrane by autologous conjunctival transplantation.” Ophthalmology 89.9 (1982): 1072-1081.

Badylak, Stephen F. “The extracellular matrix as a biologic scaffold material.” Biomaterials 28.25 (2007): 3587-3593.

Mazza, G.; Al-Akkad, W.; Telese, A.; Longato, L; Urbani, L; Robinson, B.; Hall, A. et al. “Rapid production of human liver scaffolds for functional tissue engineering by high shear stress oscillation-decellularization”. Scientific Reports 7(1):5534 (2017).

Flaupt, J.; Lutter, G.; Gorb, S. N.; Simionescu, D. T.; Frank, D.; Seiler, J.; Paur, A.; Flaben, I. “Detergent-based decellularization strategy preserves macro- and microstructure of heart valves”. Interactive Cardiovascular and Thoracic Surgery, 26(2):230-36 (2018).

Xing, Q.; Yates, K.; Tahtinen, M.; Shearier, E.; Qian, Z.; Zhao, F. “Decellularization of Fibroblast Cell Sheets for Natural Extracellular Matrix Scaffold Preparation”. TISSUE ENGINEERING: Part C, 21 (1):77-87 (2015).

Mendoza-Novelo B., Avila E. E., Cauich-Rodriguez J. V., et al. Decellularization of pericardial tissue and its impact on tensile viscoelasticity and glycosaminoglycan content. Acta Biomaterialia. 2011; 7(3):1241-1248.

Sullivan D. C., Mirmalek-Sani S.-H., Deegan D. B., et al. Decellularization methods of porcine kidneys for whole organ engineering using a high-throughput system. Biomaterials. 2012; 33(31):7756-7764.

Gilpin S E, Guyette J P, Gonzalez G, Ren X, Asara J M, Mathisen D J, Vacanti J P, Ott H C. Perfusion decellularization of human and porcine lungs: bringing the matrix to clinical scale. Cells Tissues Organs. 2012; 195(3): 222-231.

Petersen T. H., Calle E. A., Colehour M. B., Niklason L. E. Matrix composition and mechanics of decellularized lung scaffolds. Cells Tissues Organs. 2012; 195(3):222-231.

Petersen T H, Calle E A, Zhao L, Lee E J, Gui L, Raredon M B, Gavrilov K, Yi T, Zhuang Z W, Breuer C, Herzog E, Niklason LE. Tissue-engineered lungs for in vivo implantation. Science. 2010; 329(5991):538-41.

Gilpin A, Yang Y. Decellularization Strategies for Regenerative Medicine: From Processing Techniques to Applications. BioMed Research International. 2017; 2017:9831534.

Kasimir M T, Weigel G, Sharma J, Rieder E, Seebacher G, Wolner E, Simon P. The decellularized porcine heart valve matrix in tissue engineering: platelet adhesion and activation. Thrombosis and Haemostasis. 2005 94(3):562-7.

Paz A C, Kojima K, Iwasaki K, Ross J D, Canseco J A, Umezu M, Vacanti C A. Tissue Engineered Trachea Using Decellularized Aorta. Journal of Bioengineering & Biomedical Science. S2:001. doi :10.4172/2155-9538. S2-001.

Azhim A, Syazwanil N, Morimoto Y, Furukawa KS, Ushida T. The use of sonication treatment to decellularize aortic tissues for preparation of bioscaffolds. Journal of Biomaterials Applications. 2014, 29(1):130-141.

Wu X, Wang Y, Wu Q, Li Y, Li L, Tang J, Shi Y, Bu H, Bao J, Xie M. Genipin-crosslinked, Immunogen-Reduced Decellularized Porcine Liver Scaffold for Bioengineered Hepatic Tissue. Tissue Engineering and Regenerative Medicine. 2015; 12(6):417-426.

Powell H M, Boyce S T. EDC cross-linking improves skin substitute strength and stability. Biomaterials. 2006; (34):5821-7.

Zhai W, Chang J, Lin K, Wang J, Zhao Q, Sun X. Crosslinking of decellularized porcine heart valve matrix by procyanidins. Biomaterials. 2006 27(19):3684-90.

Chen Z, Wang L, Jiang H. The effect of procyanidine crosslinking on the properties of the electrospun gelatin membranes. Biofabrication. 2012 4(3):035007.

Koch H, Graneist C, Emmrich F, Till H, Metzger R, Aupperle H, Schierle K, Sack U, Boldt A. Xenogenic esophagus scaffolds fixed with several agents: comparative in vivo study of rejection and inflammation. Journal of Biomedicine and Biotechnology. 2012; 2012:948320.

Hussein K H, Park K M, Lee Y S, Woo J S, Kang B J, Choi K Y, Kang K S, Woo H M. New insights into the pros and cons of cross-linking decellularized bioartificial organs. The International Journal of Artificial Organs. 2017; 40(4):136-141.

Wassenaar, J. W., Braden, R. L., Osborn, K. G., & Christman, K. L. (2016). Modulating in vivo degradation rate of injectable extracellular matrix hydrogels. Journal of Materials Chemistry B, 4(16), 2794-2802.

Williams C, Budina E, Stoppel W L, Sullivan K E, Emani S, Emani S M, Black L D. Cardiac extracellular matrix-fibrin hybrid scaffolds with tunable properties for cardiovascular tissue engineering. Acta Biomaterialia. 2015; 14:84-95.

Therefore, from what is understood from the literature searched, no documents were found that anticipated or suggested the teachings of the present invention, thus, the solution proposed herein comprises novelty and inventive activity before the state of the art.

### SUMMARY OF THE INVENTION

In this manner, the present invention solves the problems of the state of the art from the process for obtaining the extracellular matrix from the skin of tilapia (Oreochromis niloticus) and from the extracellular matrix from the skin of tilapia itself, aiming at the applications in several sectors, such as the medical areas, chemistry, pharmacy, dentistry, veterinary, biotechnology, molecular biology and/or tissue engineering, among others.

The present invention presents as inventive concept the following objects:

In a first object, the present invention presents the process for obtaining the decellularized extracellular matrix from the animal skin comprising the steps of:

a) preparation of skin for decellularization;

b) decellularization;

c) detoxification and chemical disinfection;

d) dehydration and vacuum packaging;

e) sterilization.

In a second object, the present invention presents a decellularized extracellular matrix obtained as defined in the first object.

A third object comprises the use of the extracellular matrix from the fish skin comprising application in the medical, chemistry, pharmacy, dentistry, veterinary, biotechnology, molecular biology and/or tissue engineering areas.

A fourth object comprises a kit comprising the decellularized extracellular matrix.

These and other objects of the invention will be immediately valued by those that are skilled in the art and will be described in detail as follows.

### DETAILED DESCRIPTION OF THE INVENTION

The present invention provides a process for obtaining extracellular matrix from fish skin, more particularly from the skin of tilapia (Oreochromis niloticus) and the application thereof in several fields.

The skin of tilapia, from the histological technique of picrosirius red, comprises approximately 90.3% collagen, of which 71.4% are type I collagen and 18.9% are type III collagen.

Further, the present invention has as some of the advantages thereof:

The decellularization with a large variety of detergents and the possible combinations thereof, as well as the use of the trypsin and DNAse leading to optimum conditions for producing the extracellular matrix. In the conditions established herein, the maximum of cells is removed from the skin of the tilapia with a minimum damage to the extracellular matrix and least possible concentration of detergent. This results in a matrix with better preserved tridimensional structure and, therefore, with more capacity for stimulating the tissue regeneration. Furthermore, it will be a decellularized skin that is poor in fish cellular protein (highly immunogenic), and rich in extracellular matrix proteins (mainly collagen), little immunogenic.

The process of the present invention presents several steps of washing and successive incubations with detoxification buffer alternated with a physiological solution and ultrapure water to lessen the possibility of detergent residues and other contaminants, to guarantee the low toxicity and higher safety of the material.

Freeze-drying and vacuum packaging guarantee more stability and validity to the decellularized skin, by minimizing the humidity that is necessary for the microbial growth, hydrolysis reactions and the contact with the atmospheric oxygen. Further, by waiving the need for refrigeration of the product, there is less cost with the transport and storage of the product.

The physical and chemical modifications cited in the present invention enable the manufacture of a decellularized skin:

a) more resistance to mechanical tension, with increase of resistance to traction, specific pressure and deformation;

b) non-absorbable decellularized skin and;

c) adhesive decellularized skin.

The present invention presents the use of the Extracellular Matrix from fish skin (Oreochromis niloticus), processed in several steps, with the use of detergent varying from 0.05% to 50% or 0.5 to 150 mmol/L, DNAse and/or RNAse in concentrations varying from 0.005 μg/mL to 0.5 g/mL, protease in concentrations varying from 0.005 μg/mL to 0.5 μg/mL, crosslinking agents or crosslinking promoter agents in concentrations varying from 0.01% to 2.0% or 0.01 mg/mL to 50.0 mg/mL or 0.05 μg/mL to 500 μg/mL, with pH varying from 2.5 to 11.5 and hydrogen peroxide varying from 0.5% to 85%. The process is carried out in a Clean Room Classification environment, being subsequently carried out the dehydration, vacuum packaging, and sterilization by gamma radiation. The extracellular matrix, as herein described, can be used for a variety of applications in the health area such as, for example: rupture of diverse tissues, dermatitises, acute, chronic and traumatic lesions, battlefield wounds, necrotic wounds, lacerations, abrasions, bruises, necrotizing fasciitis, toxic epidermal necrolysis (TEN), Stevens Johnson syndrome, pressure wounds, ulcers due to venous insufficiency, arterial ulcers, diabetic or neuropathic ulcers, mixed ulcers, mucormycosis, vasculitis wounds, gangrenous pyoderma, reconstruction of the abdominal wall for hernia repair, substitute of dura-mater, dural repair, correction of myelomeningocele and encephalocele, tympanoplasty, treatment for second and third degree burns, enterocutaneous fistula, periodontal graft, inguinal hernia, rectovaginal fistula, anal fistula, eyelid reconstruction, nasal septum repair, nasosinusal repair, reconstruction of nasal and buccal lining, buccal mucous lesions, facial reconstruction, hiatal hernia, ventral hernia, rectal prolapse, Peyronie disease repair, urethra and ureter reconstruction, pelvic floor prolapse, pericardium repair, esophageal lesions due to trauma or tumor, cardiac valve reconstruction, use in cardiovascular surgeries, congenital vaginal agenesis, neovaginal construction, vagina reconstruction, sexual reassignment for transgenders, breast prosthesis wrap, fat grafting pouch, genital prolapse, eardrum reconstruction, skin lesions and surgical reconstructions in animals. Can also be used as a mesh or suture material, as raw material in the production of suture thread, or used to strengthen the mesh or suture. Can further be used to reinforce or improve the care with the wounds or associated to a support product for tissue reconstitution, such as wound support, mesh materials, bandages, or sutures. Can be used as a sling in raising the uterus and bladder and other structures. Associated or not with other materials, can be used for repair and recovering of tendons, sutures in meninges, vessel aneurysms (recovering or reconstructing the walls of the vessel). The extracellular matrix can be used by itself or incorporated with primary, permanent, stem cells, associated or not with growth factors, recombinant proteins, drugs, or natural products. In dentistry, it can be used for filling of oral mucosa, dental cavity, and alveoli.

The extracellular matrix is obtained from the skin of the tilapia (Oreochromis niloticus), being the same acquired in fish farms or tanks using a biofloc technology system, passing through a chemical and enzymatic decellularization process bleaching, as described below.

After slaughter, the skins are mechanically removed and submitted to being washed in running water, for the removal of any blood residue and placed in sterile plastic container, previously cooled at 4° C., for the transport to the laboratory, where the procedures for obtaining the extracellular matrix from the skin of tilapia are carried out:

In a first object, the present invention presents the process for obtaining the decellularized extracellular matrix from the animal skin comprising the steps of:

a) preparation of skin for decellularization;

b) decellularization;

c) detoxification and chemical disinfection;

d) dehydration and vacuum packaging;

e) sterilization.

In one embodiment, the decellularized animal skin is preferably from fish, more preferably from bony fish, optionally the fish is tilapia Oreochromis niloticus.

In one embodiment, the process comprises after step (c) the additional steps of:

i) crosslinking; and

ii) bleaching;

wherein just step (i) or (ii) may be carried out or both of them.

In one embodiment, step (b) comprises chemical and/or enzymatic decellularization, wherein the chemical decellularization is optionally assisted by microwave.

In one embodiment, step (a) comprises after obtaining and cleaning of the skin, the freezing thereof between −70° C. and −150° C. for 1 h-24 h; and thawing at 37° C. in Tris-HCl buffer or phosphate buffer saline 50-150 mmol/L, pH 6.5 to 7.5, under orbital agitation from 50 to 300 rpm, wherein this incubation with the buffer solution is repeated from 1-10 times.

In one embodiment, the chemical decellularization comprised in step (b) comprises the following sub-steps:

b1) washing the skin with physiological saline and storing in phosphate buffer saline solution or Tris-HCl or monobasic sodium phosphate/dibasic sodium phosphate or citrate/phosphate or HEPES (4-(2-hydroxyethyl)-1-piperazineethanesulfonic) acid at 0.025-0.50 mol/L, pH 6.0-8.5, having optionally added ethylenediamine tetraacetic acid (EDTA) 1.0-5.0 mmol/L and/or sodium chloride (NaCl) 0.025-0.15 mol/L and/or ammonium hydroxide (NH4OH) 0.01-1.0% (v/v), with a detergent varying in concentration from 0.01% to 50% (v/v) or 0.5 to 150 mmol/L, under agitation at 50 to 300 rpm, temperature at 20° C. to 40° C., and time from 30 min to 24 h, with 1 to 5 optional changes of buffer with detergent;

b2) removal of the skin from the b1 solution and storing in the same solution without detergent, for exhaustive washing, where it must remain under orbital agitation from 50 to 300 rpm for 30 min to 24 h, at a temperature of 20° C. to 40° C., with intervals for 2 to 7 solution changes.

In one embodiment, the said detergent in b1 is comprised from the group: sodium dodecyl sulfate (sodium lauryl sulfate), sodium deoxycholate, t-octylphenoxypolyethoxyethanol (Triton X-100), 3-[(3-cholamidopropyl) dimethylammonium]-1-propanesulfonic (CHAPS) 4-nonylphenyl-polyethylenoglycol (Nonidet P-40 substitute or NP40) or polysorbate 20 (Tween 20) or combinations thereof.

In one embodiment, the enzymatic decellularization comprised in step (b) comprises the enzymatic decellularization with DNAse, RNAse, and/or protease(s), optionally the enzymatic decellularization comprises the combinations thereof, wherein, if the combination is made, initially the treatment is optionally made with nucleases and subsequently the treatment with proteases is carried out.

In one embodiment, the enzymatic decellularization in step (b) comprises the following sub-steps:

b3) for the incubation with DNAse and/or RNAse, the exhaustive washing with the said solution in (b2) is replaced by the phosphate buffer saline solution or Tris-HCl or monobasic sodium phosphate/dibasic sodium phosphate or citrate/phosphate or HEPES (4-(2-hydroxyethyl)-1-piperazineethanesulfonic) acid at 0.025-0.50 mol/L for incubation with DNAse or 0.01-0.50 mol/L for incubation with RNAse, pH 6.0-8.5, additionally adding MgCl2 at 0.5-10.0 mmol/L, NaCl at 0.5-50.0 mmol/L and CaCl2 at 0.5-10.0 mmol/L, where the skin must remain under orbital agitation from 50 to 300 rpm for 30 min to 24 h, at a temperature of 20° C. to 40° C., with intervals for 2 to 7 solution changes;

b4) storing the skins in the (b3) solution with added DNAse or RNAse in concentration varying from 0.005 μg/mL to 0.5 g/mL, where the skin must remain under orbital agitation from 50 to 300 rpm for 30 min to 24 h, at a temperature of 20° C. to 40° C.;

b5) after the treatment with DNAse and/or RNAse, a new exhaustive washing of the skin is carried out by removal of the (b4) solution and

storing the said (b2) solution, where the skin must remain under orbital agitation from 50 to 300 rpm for 30 min to 24 h, at a temperature of 20° C. to 40° C., with intervals for 2 to 7 solution changes;

b6) for the incubation with protease, the exhaustive washing with the said solution in (b2) is replaced with the phosphate buffer saline solution or Tris-HCl or monobasic sodium phosphate/dibasic sodium phosphate or citrate/phosphate or HEPES (4-(2-hydroxyethyl)-1-piperazineethanesulfonic) acid, at 0.025-0.50 mol/L, pH 6.0-8.5 optionally added MgCl2 at 0.5-10.0 mmol/L, NaCl at 0.5-50.0 mmol/L, CaCl2 at 0.5-10.0 mmol/L and ethylenediamine tetraacetic acid (EDTA) 1.0-5.0 mmol/L, with the protease(s) chosen in concentrations varying from 0.005 μg/mL to 0.5 μg/mL, where the skin must remain under orbital agitation from 50 to 300 rpm for 30 min to 24 h, at a temperature of 20° C. to 40° C.;

b7) after the treatment with protease, the new exhaustive washing must be carried out with the removal of the (b6) solution and storing of the skin in the phosphate buffer solution or Tris-HCl or monobasic sodium phosphate/dibasic sodium phosphate or citrate/phosphate or 4-(2-hydroxyethyl)-1-piperazineethanesulfonic) (HEPES) acid at 0.025-0.50 mol/L, pH 6.0 -8.5, with added CaCl2 100.0-200.0 mg/L and MgCl2 100.0-150.0 mg/L, where the skin must remain under orbital agitation from 50 to 300 rpm for 30 min to 24 h, at a temperature of 20° C. to 40° C., with intervals for 2 to 7 solution changes.

In one embodiment, the said protease in (b6) is chosen from the group selected between: trypsin and/or subtilisin and/or collagenase and/or dispase and/or bromelain and/or pepsin or combinations thereof.

In one embodiment, the step (c) comprises the following sub-steps:

c1) incubation of the skin in sterile container containing bactericidal agent at 0.005-1.0% (m/v), for 15-60 minutes, under agitation from 50 to 300 rpm, at a temperature of 20° C. to 40° C., followed by rising in ultrapure sterile water in the same container for 15-60 minutes, in the same conditions of agitation and temperature, from one to ten repetitions; wherein the bactericidal agent is selected from the group that comprises: chlorhexidine digluconate, sodium chlorite, cetylpyridinium chloride, chloramine T, sodium dichloroisocyanurate, optionally the bactericidal agent is the chlorexidine digluconate;

c2) incubation of the skin in sterile container containing acetic acid/acetate buffer or glycine/HCl or citric acid/citrate or monobasic sodium phosphate/dibasic sodium phosphate at 0.025-0.50 mol/L, pH 3.0-6.0, for 30-120 minutes, under agitation from 50 to 300 rpm, at a temperature from 20° C. to 40° C., followed by incubation with ultrapure sterile water in the same container for 15-60 minutes, in the same conditions of agitation and temperature, in one to ten repetitions

c3) incubation of the skin in sterile container containing Tris-HCl buffer or phosphate saline buffer or monobasic sodium phosphate/dibasic sodium phosphate or citrate/phosphate or 4-(2-hydroxyethyl)piperazine-1-ethanesulfonic acid (HEPES) at 0.025-0.50 mol/L, pH 6.0-8.5, for 30 minutes-24 hours, under agitation from 50 to 300 rpm, at a temperature from 20° C. to 40° C., in five to thirty repetitions.

In one embodiment, the chemical decellularization comprised in step (b) can be assisted with microwave with a frequency between 1.0 GHz and 3.0 GHz or between 100 kHz to 300 kHz, under continuous cooling of the solution between 4° C. and 18° C., under agitation from 50 to 200 rpm during the treatment times.

In one embodiment, the additional step (i) comprises:

incubation of the skin in sterile container containing Hanks solution or phosphate buffer saline solution or 4-(2-hydroxyethyl)piperazine-1-ethanesulfonic acid (HEPES) or 2-(N-morpholino)ethanesulfonic) acid (MES) at 0.015-0.50 mol/L, pH 3.0-8.5, for 30 to 360 minutes, under agitation from 50 to 300 rpm, at a temperature from 20° C. to 40° C., in one to five repetitions;

incubation of the skin in sterile container containing Hanks solution or phosphate buffer saline solution or 4-(2-hydroxyethyl)piperazine-1-ethanesulfonic acid (HEPES) or 2-(N-morpholino)ethanesulfonic) acid (MES) at 0.015-0.50 mol/L, pH 3.0-8.5, with addition of crosslinking reagent at 0.01%-2.0% or 0.01-50.0 mg/mL or 0.05-500 μg/mL for 30 minutes to 24 hours, under agitation from 50 to 300 rpm, at a temperature from 20° C. to 40° C., in one to five repetitions;

incubation of the skin in sterile container containing physiological solution for 30 minutes to 24 hours, under agitation from 50 to 300 rpm, at a temperature from 20° C. to 40° C., in one to fifteen repetitions.

In one embodiment, the said crosslinking reagent or crosslinking promoter agent in e2 comprises being selected from the group: glutaraldehyde (GDA) and/or genipin (GP) and/or N-hydroxysuccinimide (NHS) and/or N-(3-dimethylaminopropyl)-N-ethylcarbodiimide (EDC) and/or procyanidin (PA) and/or transglutaminase (TG) or combinations thereof.

In one embodiment, the additional step (ii) comprises:

incubated skin with a hydrogen peroxide solution, under agitation from 50 to 300 rpm, temperature from 20° C. to 40° C., for a period from 30 min to 24 h;

exhaustive washing of the skin with ultrapure water;

incubation of the skins in saline phosphate buffer for a period from 30 min to 24 h;

exhaustive washing with sterile ultrapure water and incubation with physiological solution, under agitation from 50 to 300 rpm, temperature from 20° C. to 40° C., with intervals for solution change, for a period from 1 h to 24 h.

In one embodiment, the step (d) occurs in a freeze-dryer in a range from −30° C. to −80° C., with internal pressure lower than 50 μmHg, optionally between 30 and 35 μmHg and time from 2 to 24 h, followed by vacuum packaging of the skins in sterilized plastic packaging with thickness from 0.15 to 0.40 pm.

In one embodiment step (e) comprises the complementary gamma-ray sterilization in an irradiator of Cobalt-60, with dosages varying between 5 to 50 kGy.

In a second object, the present invention presents an extracellular matrix obtained as defined in the first object.

In one embodiment, the decellularized extracellular matrix is comprised by a mesh or suture material, as raw material in the production of suture thread, or used to strengthen mesh or suture material, incorporated with primary, permanent, stem cells, associated or not to growth factors, recombinant proteins, drugs or natural products.

A third object comprises the use of the extracellular matrix from the fish skin comprising application in the medical, chemistry, pharmacy, dentistry, veterinary, biotechnology, molecular biology and/or tissue engineering areas.

In one embodiment, there is comprised the use for application in rupture of diverse tissues, dermatitises, acute, chronic and traumatic lesions, battlefield wounds, necrotic wounds, lacerations, abrasions, bruises, necrotizing fasciitis, toxic epidermal necrolysis (TEN), Stevens Johnson syndrome, pressure wounds, ulcers due to venous insufficiency, arterial ulcers, diabetic or neuropathic ulcers, mixed ulcers, mucormycosis, vasculitis wounds, gangrenous pyoderma, reconstruction of the abdominal wall for hernia repair, substitute of dura-mater, dural repair, correction of myelomeningocele and encephalocele, tympanoplasty, treatment for second and third degree burns, enterocutaneous fistula, periodontal graft, inguinal hernia, rectovaginal fistula, anal fistula, eyelid reconstruction, nasal septum repair, nasosinusal repair, reconstruction of nasal and buccal lining, buccal mucous lesions, facial reconstruction, hiatal hernia, ventral hernia, rectal prolapse, Peyronie disease repair, urethra and ureter reconstruction, pelvic floor prolapse, pericardium repair, esophageal lesions due to trauma or tumor, cardiac valve reconstruction, use in cardiovascular surgeries, congenital vaginal agenesis, neovaginal construction, vagina reconstruction, sexual reassignment for transgenders, breast prosthesis wrap, fat grafting pouch, genital prolapse, eardrum reconstruction, skin lesions and surgical reconstructions in animals.

In one embodiment, the present invention comprises the use of the extracellular matrix as a mesh or suture material, as raw material in the production of suture thread, or used to strengthen mesh or suture material.

In one embodiment, the use of the extracellular matrix is comprised as a material to reinforce or improve the care with the wounds or associated to a support product for tissue reconstitution, such as wound support, web materials, bandages or sutures.

In one embodiment, the present invention comprises the use of the extracellular matrix as a sling in raising the uterus and bladder and other structures.

In one embodiment, the present invention comprises the use of the extracellular matrix associated or not with other materials, in the repair and recovering of tendons, sutures in meninges, vessel aneurysms (recovering or reconstructing the walls of the vessel).

In one embodiment, the extracellular matrix can optionally be used by itself or incorporated with primary, permanent, stem cells, associated or not with growth factors, recombinant proteins, drugs or natural products.

In one embodiment, in dentistry, it can be used for filling or oral mucosa, dental cavity and alveoli.

As a fourth object, the present invention presents a kit, comprising the decellularized extracellular matrix, as defined in the second object and the concretization thereof.

### EXAMPLES

The examples shown herein have the purpose only of exemplifying one of the countless ways to perform the invention, without however limiting the scope of the same.

### Example 1—Process for Obtaining the Extracellular Matrix From Tilapia

The tilapias (Oreochromis niloticus) are acquired in fish farms or tanks using a biofloc technology system, passing through a chemical and enzymatic decellularization process, bleaching, dehydration, vacuum packaging, and sterilization, as described below.

After slaughter, the skins are mechanically removed and submitted to being washed in running water, for the removal of any blood residue and placed in sterile plastic container, previously cooled at 4° C., for the transport to the laboratory.

### Step 1—Preparation of Skin for Decellularization

In the laboratory, the excess muscle is removed, the edges are cut and washing is carried out with a sterile physiological solution (NaCl solution at 0.9%);

The skins are frozen for 1 h-24 h (optionally 16 h) between −70° C. and −150° C. (optionally −80° C.) and thawed at 37° C. in a Tris-HCl solution bath or phosphate saline buffer, under orbital agitation from 50 to 300 rpm (optionally 100 rpm). This cycle is repeated from 1 to 10 times (optionally 1).

### Step 2—Chemical and Enzymatic Decellularization Process

In step 2, the skins are removed from the Tris-HCl solution or phosphate buffer saline solution, washed with physiological saline solution and stored in containers containing a phosphate saline solution with sodium dodecyl sulfate, varying from 0.01% to 50% (optionally 0.5%), under agitation from 50 to 300 RPM (optionally 130 RPM), temperature from 20° C. to 40° C. (optionally 37° C.), and time from 30 min to 6 h (optionally 2 h). Next, the skins are submitted to the exhaustive washing in the same solution, however, without detergent.

As alternative or additional incubation some additional steps for step 2 are presented below:

Additional step 2A: an alternative or additional incubation for step 2 can be carried out with the sodium deoxycholate detergent varying from 0.01% to 50% (optionally 4%) in phosphate buffer saline solution, under agitation from 50 to 300 RPM (optionally 130 RPM), temperature from 20° C. to 40° C. (optionally 37° C.), and time from 30 min to 24 h (optionally 1 h). This step must be followed by exhaustive washing in monobasic sodium phosphate buffer solution/dibasic phosphate sodium (NaH2PO4Na2HPO4) 0.1 mol·L−1; MgCl2 10.0 mmol·L−1, NaCl 5.0 mmol·L−1 and CaCl2 2.5 mmol·L−1, pH 6.5, followed by treatment with DNAse and RNAse, in concentrations of 0.01 μg·mL−1 to 0.5 g·mL−1 (optionally 0.μg·mL−1) in the same buffer, under agitation from 50 to 300 RPM (optionally 130 RPM), temperature from 20° C. to 40° C. (optionally 37° C.), and time from 30 min to 24 h (optionally 4 h).

Additional step 2B: the Triton X-100 detergent can also be used in the alternative or additional incubation to step 2, in concentration varying from 0.01% to 50% (optionally 1%), together with ammonium hydroxide in concentration varying from 0.01% to 1.0% (optionally 1.0%), in phosphate buffer saline solution, under agitation from 50 to 300 RPM (optionally 130 RPM), temperature from 20° C. to 40° C. (optionally 37° C.), and time from 30 min to 24 h (optionally 6 h). This step can be followed by exhaustive washing and treatment with DNAse and RNAse, as described previously.

Additional step 2C: the detergent 3-[(3-cholamidopropyl)dimethylammonium]-1-propanesulfonate (CHAPS) can also be used in the alternative or additional incubation in step 2 in a concentration varying from 0.5 to 150 mmol·L−1, (optionally 8.0 mmol·L−1) in a solution of NaCl 0.1 mol·L−1; EDTA 2.5 mmol·L−1, under agitation from 50 to 300 RPM (optionally 130 RPM), temperature from 20° C. to 40° C. (optionally 37° C.), and time from 30 min to 24 h (optionally 4 h). This step can be followed by exhaustive washing and treatment with DNAse and RNAse, as described previously.

Additional step 2D: the detergent Nonidet P-40 (NP40) can also be used in the alternative or additional incubation in step 2 in a concentration varying from 0.01% to 50% (optionally 0.05%), in a phosphate buffer saline solution, under agitation from 50 to 300 RPM (optionally 130 RPM), temperature from 4° C. to 37° C. (optionally 37° C.), and time from 2 h to 24 h (optionally 16 h). This step can be followed by exhaustive washing and treatment with DNAse and RNAse, as described previously.

Additional step 2E: the detergent Tween 20 (Polysorbate 20) can also be used in the alternative or additional incubation in step 2 in a concentration varying from 0.01% to 10% (optionally 3%), in buffer Tris-HCl buffer 50 mmol·L−1, pH 7.5, NaCl 0.15 mol·L−1, under agitation from 50 to 300 RPM (optionally 130 RPM) temperature from 4° C. to 37° C. (optionally 37° C.) , and time from 2 h to 24 h (optionally 24 h). This step can be followed by exhaustive washing and treatment with DNAse and RNAse, as described previously.

Additional step 2F: different combinations of the detergents used in steps 2A-2E can be used. This step can be followed by exhaustive washing and treatment with DNAse and RNAse, as described previously.

Additional step 2G: the decellularization with detergents can be assisted with microwave with frequency between 1.0 GHz and 3.0 GHz, under continuous cooling of the solution between 4° C. and 18° C., under agitation from 50 to 200 RPM, in the treatment times described in additional steps 2A-2E or reduced.

### Steps 3 and 4—Continuation of the Decellularization Process

In step 3, the skins are removed from the previous solution and stored in a phosphate buffer saline solution with EDTA 5.0 mmol·L−1, pH 8.5, with subtilisin 0.005 μg·mL−1 to 0.5 g·mL−1 (optionally 0.2 mg·mL−1) remaining from 2 to 24 h (optionally 1 h), under agitation from 50 to 300 RPM (optionally 130 RPM), at a temperature from 20° C. to 60° C. (optionally 50° C.).

As alternative or additional incubation in step 3 some additional steps are presented below:

Additional step 3A: the collagenase protease can also be used in the alternative or additional incubation in step 3 in a concentration varying from 0.005 μg·mL−1 to 0.5 g·mL−1 (optionally 0.5 mg·mL−1 1), in Tris-HCl buffer 50 mmol·L−1, pH 7.5, NaCl 0.15 mol·L−1, pH 6.0, under agitation from 50 to 300 RPM (optionally 130 RPM), temperature from 20° C. to 40° C. (optionally 37° C.), and time from 2 h to 24 h (optionally 2 h).

Additional step 3B: the trypsin protease can also be used in the alternative or additional incubation in step 3 in a concentration varying from 0.005 μg·mL−1 to 0.5 g·mL−1 (optionally 0.5 mg·mL−1), in phosphate buffer 0.05 mmol·L−1 with EDTA 5.0 mmol·L−1, pH 8.5, under agitation from 50 to 300 RPM (optionally 130 RPM), temperature from 20° C. to 40° C. (optionally 37° C.), and time from 2 h to 24 h (optionally 2 h).

Additional step 3C: the dispase protease can also be used in the alternative or additional incubation in step 3 in a concentration varying from 0.005 μg·mL−1 to 0.5 g·mL−1 (optionally 0.2 mg·mL−1), in 4-(2-hydroxyethyl)piperazine-lethanesulfonic acid (HEPES) 0.05 mmol·L−1, pH 7.5, under agitation from 50 to 300 RPM (optionally 130 RPM), temperature from 20° C. to 40° C. (optionally 37° C.), and time from 2 h to 24 h (optionally 2 h).

Additional step 3D: the bromelain protease can also be used in the alternative or additional incubation in step 3 in a concentration varying from 0.005 μg·mL−1 to 0.5 g·mL−1 (optionally 0.1 mg·mL−1), in phosphate buffer 0.05 mmol·L−1 with EDTA 1.0 mmol·L−1, pH 6.5, under agitation from 50 to 300 RPM (optionally 130 RPM) temperature from 20° C. to 40° C. (optionally 37° C.), and time from 2 h to 24 h (optionally 2 h).

Additional step 3E: the pepsin protease can also be used in the alternative or additional incubation in step 3 in a concentration varying from 0.005 μg·mL−1 to 0.5 g·mL−1 (optionally 0.5 mg·mL−1), in citrate/phosphate buffer 0.05 mmol·L−1, pH 4.0, under agitation from 50 to 300 RPM (optionally 130 RPM), temperature from 20° C. to 40° C. (optionally 37° C.), and time from 2 h to 24 h (optionally 2 h).

In step 4, the skins are stored in flasks containing the Tris-HCl solution 0.025 mol·L−1 to 0.5 mol·L−1 (optionally 0.050 mol·L−1) CaCl2 100.0 to 200.0 mg·mL−1(optionally 140.0 mg·mL−1) and MgCl2 100.0 to 150.0 mg·mL−1 (optionally 98.0 mg·mL−1) pH of 6.0 to 8.5 (optionally 7.4), under agitation from 50 to 300 RPM (optionally 130 RPM), temperature from 20° C. to 40° C. (optionally 37° C.), with intervals for solution change, for a period from 2 h to 24 h (optionally 2 h), with intervals for 2 to 7 (optionally 3) solution changes.

### Steps 5 To 7—Chemical Detoxification and Disinfection Process

In step 5 the skins are removed from the previous solution and stored in a chlorhexidine digluconate solution with concentration between 0.005 and 1.0% (optionally 0.5%) (m/v), for 15 to 60 minutes (optionally 60 minutes), under agitation from 50 to 300 RPM (optionally 250 RPM) and temperature from 20° C. to 40° C. (optionally 37° C.), followed by incubation with sterile ultrapure water for 15 to 60 minutes (optionally 60 minutes), in the same agitation and temperature conditions, in one to ten repetitions.

In step 6 the skins are removed from the previous solution and stored in an acetic acid/acetate buffer 0.1 mol·L−1, pH 3.0, for 30 to 120 minutes (optionally 60 minutes), under agitation from 50 to 300 RPM (optionally 250 RPM) and temperature from 20° C. to 40° C. (optionally 37° C.), to then are incubated with sterile ultrapure water in the same container for 15-60 minutes (optionally 60 minutes), in the same agitation and temperature conditions, in one to ten repetitions (optionally three repetitions).

As alternative or additional incubation some additional steps for step 6 are presented below:

Additional step 6A: the citric acid/citrate buffer 0.25 mol·L−1, pH 5.0, can also be used in the alternative or additional incubation in step 6, under agitation from 50 to 300 RPM (optionally 250 RPM) temperature from 20° C. to 40° C. (optionally 37° C.), and time from 30 to 120 (optionally 60) minutes.

Additional step 6B: the glycine buffer/HCl 0.25 mol·L−1, pH 6.0, can also be used in the alternative or additional incubation in step 6, under agitation from 50 to 300 RPM (optionally 250 RPM) temperature from 20° C. to 40° C. (optionally 37° C.), and time from 30 to 120 (optionally 60) minutes.

Additional step 6C: the monobasic sodium phosphate buffer/dibasic sodium phosphate buffer 0.050 mol·L−1, pH 4.0, can also be used in the alternative or additional incubation in step 6, under agitation from 50 to 300 RPM (optionally 250 RPM) temperature from 20° C. to 40° C. (optionally 37° C.), and time from 30 to 120 (optionally 60) minutes.

In step 7, the skins are removed from the previous solution and stored in a Tris-HCl buffer solution 0.05 mol·L−1, pH 8.5, for 30 minutes to 24 hours (optionally 60 minutes), under agitation from 50 to 300 RPM (optionally 250 RPM) and temperature from 20° C. to 40° C. (optionally 37° C.), in five to thirty repetitions (optionally five repetitions).

As alternative or additional incubation some additional steps for step 7 are presented below:

Additional step 7A: the phosphate saline buffer solution 0.10 mol·L−1, pH 6.5, can also be used in the alternative or additional incubation in step 7, under agitation from 50 to 300 RPM (optionally 250 RPM), temperature from 20° C. to 40° C. (optionally 37° C.), and time from 30 minutes to 24 hours (optionally 60 minutes).

Additional step 7B: the monobasic sodium phosphate buffer/dibasic sodium phosphate buffer 0.05 mol·L−1, pH 7.5, can also be used in the alternative or additional incubation in step 7, under agitation from 50 to 300 RPM (optionally 250 RPM), temperature from 20° C. to 40° C. (optionally 37° C.), and time from 30 minutes to 24 hours (optionally 60 minutes).

Additional step 7C: the 4-(2-hydroxyethyl)piperazine-1-ethanesulfonic acid (HEPES) 0.25 mol·L−1, pH 7.5, can also be used in the alternative or additional incubation in step 7, under agitation from 50 to 300 RPM (optionally 250 RPM), temperature from 20° C. to 40° C. (optionally 37° C.), and time from 30 minutes to 24 hours (optionally 60 minutes).

Additional step 7D: the citrate/phosphate buffer 0.15 mol·L−1, pH 6.0, can also be used in the alternative or additional incubation in step 7, under agitation from 50 to 300 RPM (optionally 250 RPM), temperature from 20° C. to 40° C. (optionally 37° C.), and time from 30 minutes to 24 hours (optionally 60 minutes).

### Steps 8 and 9—Crosslinking Process (Addition of Chemical Crosslinking)

In step 8, the skins are exhaustively washed and incubated with Hanks solution or phosphate buffer solution or 4-(2-hydroxyethyl)piperazine-1-ethanesulfonic acid (HEPES) or 2-(N-Morpholino)ethanesulfonic acid (MES) at 0.015-0.50 mol·L−1 (optionally 50 mmol·L−1 and 0.2 mol·L−1, respectively), pH 3.0-8.5 (optionally 7.4 and 5.0, respectively), under agitation from 50 to 300 RPM (optionally 150 RPM), temperature from 20° C. to 40° C. (optionally 37° C.), with intervals for solution change, for a period from 30 min to 24 h (optionally 1 h). The solution that is selected for incubation of the skins must be the same that is used as reaction means for the following crosslinking.

In step 9, the skins are incubated with the crosslinking promoter agent or glutaraldehyde crosslinking reagent (GDA), in concentrations from 0.01% to 2% (optionally 0.625%), in HEPES buffer 50 mmol·L−1 (pH 7.4), sunder agitation from 50 to 300 RPM (optionally 150 RPM), temperature from 20° C. to 40° C. (optionally 37° C.), and time from 2 h to 72 h (optionally 2 h). This step must be followed by exhaustive washing in physiological solution.

As alternative or additional incubation some additional steps for step 9 are presented below:

Additional step 9A: an alternative or additional incubation in step 9 can be carried out with a crosslinking agent or genipin crosslinking reagent (GP), in concentrations from 0.3% to 1% (optionally 0.5%), in HEPES buffer 50 mmol·L−1 (pH 7.4), under agitation from 50 to 300 RPM (optionally 150 RPM), temperature from 20° C. to 40° C. (optionally 37° C.), and time from 2 h to 72 h (optionally 3 h). This step must be followed by exhaustive washing in physiological solution.

Additional step 9B: an alternative or additional incubation in step 9 can be carried out with a crosslinking agent or crosslinking reagent N-hydroxysuccinimide (NHS) in concentrations from 0.05% to 0.2% (optionally 0.1%), in MES buffer 0.2 mol·L−1 (pH 5.0), under agitation from 50 to 300 RPM (optionally 150 RPM), temperature from 20° C. to 40° C. (optionally 37° C.), and time from 2 h to 24 h (optionally 2 h). This step must be followed by exhaustive washing in physiological solution.

Additional step 9C: an alternative or additional incubation in step 9 can be carried out with a crosslinking agent or crosslinking reagent N-(3-dimethylaminepropyl)-N-ethylcarbodiimide (EDC), in concentrations from 0.05% to 0.5% (optionally 25%), in MES buffer 0.2 mol·L−1 (pH 5.0), under agitation from 50 to 300 RPM (optionally 150 RPM), temperature from 20° C. to 40° C. (optionally 37° C.), and time from 2 h to 24 h (optionally 2 h). This step must be followed by exhaustive washing in physiological solution.

Additional step 9D: an alternative or additional incubation in step 9 can be carried out with a crosslinking agent or crosslinking reagent procyanidin (PA), in concentrations from 1.0 to 50.0 mg·mL−1 (optionally 1.0 mg·mL−1) in D-Hanks solution (pH 7.4), under agitation from 50 to 300 RPM (optionally 150 RPM), temperature from 20° C. to 40° C. (optionally 37° C.), and time from 2 h to 24 h (optionally 16 h). This step must be followed by exhaustive washing in physiological solution.

Additional step 9E: an alternative or additional incubation in step 9 can be carried out with a crosslinking agent or crosslinking reagent transglutaminase (TG), in concentrations from 1.0 to 200 μg·mL−1 (optionally 0.1 mg·mL−1), in saline phosphate buffer (pH 6.0, under agitation from 50 to 300 RPM (optionally 150 RPM), temperature from de 20° C. to 40° C. (optionally 37° C.), and time from 2 h to 24 h (optionally 2 h). This step must be followed by exhaustive washing in physiological solution.

### Steps 10 and 11: Bleaching

In step 10 the skins are incubated with hydrogen peroxide solution 10% under agitation from 50 to 300 RPM (optionally 150 RPM), temperature from 20° C. to 40° C. (optionally 37° C.), for 30 minutes. After the incubation with hydrogen peroxide, the matrix is incubated three times with sterile ultrapure water. Once the washing with ultrapure water is concluded, the matrixes are added to 50 mL sterile phosphate saline buffer to incubate for a period from 30 min to 24 h (optionally 2 h).

After the incubation with the phosphate saline buffer, a new washing of the skins was carried out for 30 min in 50 mL of sterile ultrapure water, at a temperature from 37° C. and 120 rpm.

In step 11, the skins are exhaustively washed and incubated with physiological solution, under agitation from 50 to 300 RPM (optionally 250 RPM), temperature from 20° C. to 40° C. (optionally 37° C.), with intervals for solution changes, for a period from 1 h to 24 h (optionally 1 h);

### Step 12: Dehydration

In step 12, the skins are dehydrated at low temperatures and low pressures and, subsequently vacuum packaged in appropriate plastic packaging. This step was executed with a freeze-dryer in a range from −30° C. to −80° C., with inner pressure lower than 50 μmHg (optionally in the range from 30 to 35 μmHg, the value being optionally 30 μmHg) and time from 2 to 24 h (optionally 3 h 30 min), followed by vacuum packaging of the skins in sterilized plastic packaging having a thickness from 0.15 to 0.40 pm.

### Step 13: Sterilization

In step 13 the complementary sterilization by gamma rays is carried out in a Cobalt 60 radiator, with dosages that vary between 5 to 50 kGy (optionally 25 kGy), depending on the Bioburden levels (microbial count).

### Example 2—Applications of the Decellularized Extracellular Matrix

The extracellular matrix, by interacting with tissues, promotes the acceleration of the healing and repair processes (due to the action of the Type I Collagen that exists in the histological structure thereof).

Further, the decellularized skin of the present invention can be modified to present the following properties:

a) Decellularized skin that is more resistant to mechanical tension: for applications that require higher mechanical resistance (ex.: hernia and tendon repair), the decellularized skin can be pressed with other sheets of the same type of extracellular matrix (by vacuum or controlled atmosphere). The biomaterial that is resultant from the pressing will have several layers of extracellular matrix and will present an increase in the traction resistance, specific pressure and deformation. The pressed sheets can be positioned in orientations that are perpendicular to each other (according to the direction of the collagen fibers), for the resultant biomaterial to offer mechanical resistance both in the longitudinal direction as transversally. To improve the adhesion between the layers during pressing, a biological glue can be added between them (fibrin, hyaluronic acid, chitosan) or, as is the case, a crosslinking inducer agent (glutaraldehyde, genipin, procyanidine, transglutaminase, N-hydroxysuccinimide, N-(3-dimethylaminepropryl)-N-ethylcarbodiimide, N-hydroxysuccinimide-polyethyleneglycol]

b) Non-absorbable decellularized skin: in applications that require long-term stability of the biomaterial used in surgeries, the outer faces of the decellularized extracellular skin can be treated with crosslinking agents (glutaraldehyde, genipin, procyanidine, transglutaminase, N-hydroxysuccinimide, N-(3-dimethylaminepropryl-N-ethylcarbodiimide, N-hydroxysuccinimide -polyethyleneglycol]. The outer faces of the biomaterial are those that come into direct contact with the tissues of the host organism. The formation of crosslinks in these faces inhibits the recognition of the protein structure of the decellularized skin by host enzymes. Consequently, the reabsorption of the decellularized skin is inhibited. This characteristic is desirable, for example, in tendon replacements, vessels and artificially constructed cardiac valves, which must remain integral within the receptor organism for several years.

c) Adhesive decellularized skin: some surgical applications require that the biomaterial used be capable of quickly sealing the lesions under repair, to avoid loss of body fluids (ex.: hemostatic repairs and durals in general). For these cases, it is possible to make one of the faces of the decellularized skin readily adhesive. This face of the matrix will be employed in the immediate occlusion of the lesion/incision. To obtain this biomaterial, after the decellularization, the future adhesive face must be treated with substances that make it reactive [N-hydroxysuccinimide -polyethyleneglycol, N-(3-dimethylaminepropyl)-N-ethylcarbodiimide, N-hydroxysuccinimide] or adhesive (hyaluronic acid, chitosan and hydroxymethylpropylcellulose), while the other face remains without any additional treatment. The reactivity or adherence will allow the adhesive face to promptly fix to the site of the lesion/incision, immediately interrupting the fluid overflow.

d) The associations of the decellularized skin to drugs and bio-drugs will result in biomaterials with new properties that are useful for use as a medical device. That is, the associations can grant extra properties to the decellularized extracellular matrix or potentialize the regenerative stimulation thereof. For example, the decellularized skin can be used in the repair of a lesion where intense angiogenesis is necessary for the recovery of the afflicted tissues. In this case, the matrix that is used could have been previously associated with a pro-angiogenic substance, such as the (vascular-endothelial growth factor) VEGF. Therefore, apart from the expected stimulation to the regeneration of the filling tissues, the matrix will also grant the intense formation of new blood vessels (angiogenesis), according to the specific need of the afflicted region. Associations with antibodies to avoid the contamination with bacteria, or with pain relievers to reduce the pain of the patient are also possible.

### Example 3—Toxicity Test

The decellularized matrix from skin of tilapia was tested and approved as to the in vitro toxicity, according to the guidelines from ISO 10993-5, since it provided higher cell viability than 75% in the cytotoxicity test by extraction with lineage L929 (murine fibroblast). Tests in rats are being carried out at this time.

Those skilled in the art will value the knowledge presented herein and can reproduce the invention in the embodiments presented and in other variants and alternatives, covered by the scope of the following claims.

1. Process for obtaining decellularized extracellular matrix from animal skin, characterized by comprising the steps of:
a) preparation of skin for decellularization;
b) decellularization;
c) detoxification and chemical disinfection;
d) dehydration and vacuum packaging;
e) sterilization.

2. Process, according to claim 1, characterized in that the animal skin is from fish, optionally the fish is tilapia Oreochromis niloticus.

3. Process, according to claim 1, characterized by comprising after step (c) the additional steps of:
i) crosslinking; and
ii) bleaching;
wherein only step (i) or (ii) can be carried out or both.

4. Process, according to claim 1, characterized in that step (b) comprises chemical and/or enzymatic decellularization, wherein the chemical decellularization is optionally assisted by microwave.

5. Process, according to claim 1, characterized in that step (a) comprises, after obtaining and cleaning the skin, the freezing thereof between −70° C. and −150° C. for 1 h-24 h; and thawing at 37° C. in Tris-HCl buffer or phosphate saline buffer 50-150 mmol/L, pH 6.5 to 7.5, under orbital agitation from 50 to 300 rpm, wherein this incubation with the buffer solution is repeated from 1-10 times.

6. Process, according to claim 1 or 4, characterized in that the chemical decellularization comprised in step (b) comprises the sub-steps:
b1) washing the skin with physiological saline solution and storing in phosphate saline buffer solution or Tris-HCl or monobasic sodium phosphate/dibasic sodium phosphate or citrate/phosphate or 4-(2-hydroxyethyl)piperazine-1-ethanesulfonic acid at 0.025-0.50 mol/L, pH 6.0-8.5, with optional addition of ethylenediaminetetraacetic acid 1.0-5.0 mmol/L and/or sodium chloride 0.025-0.15 mol/L and/or ammonium hydroxide 0.01-1.0% (v/v), with a detergent in concentration varying from 0.01% to 50% (v/v) or 0.5 to 150 mmol/L, under agitation from 50 to 300 rpm, temperature from 20° C. to 40° C., and time from 30 min to 24 h, with 1 to 5 optional changes of buffer with detergent;
b2) removal of the skin of the b1 solution and storing in the same solution without detergent, for washing, where it must remain under orbital agitation from 50 to 300 rpm for 30 min to 24 h, at a temperature from 20° C. to 40° C., with intervals for 2 to 7 solution changes;
wherein the said detergent comprises being from the group: sodium sulfate dodecyl, t-octylphenoxypolyethoxyethanol, 3-[(3-cholamidopropyl)dimethylammonium]-1-propanesulfonate, 4-nonilphenyl-polyethyleneglycol or polysorbate 20 or combinations thereof.

7. Process, according to claim 4 or 6, characterized in that the chemical decellularization comprised in step (b) is optionally assisted by microwave with frequency between 1.0 GHz and 3.0 GHz or between 100 kHz to 300 kHz, under continuous cooling of the solution between 4° C. and 18° C., under agitation from 50 to 200 rpm during the treatment times; and by the enzymatic decellularization comprised in step (b) comprising the enzymatic decellularization with DNAse, RNAse and/or protease, optionally the enzymatic decellularization comprises the combination of same, wherein initially it is optionally treated with nucleases and subsequently the treatment with proteases is carried out.

8. Process, according to claim 4 or 7, characterized in that the enzymatic decellularization comprised in step (b) comprises the following sub-steps:
b3) for the incubation with DNAse and/or RNAse, the exhaustive washing with the said solution in (b2) is replaced by the phosphate buffer saline solution or Tris-HCl or monobasic sodium phosphate/dibasic sodium phosphate or citrate/phosphate or HEPES (4-(2-hydroxyethyl)-1 -piperazine-ethanesulfonic) acid at 0.025-0.50 mol/L for incubation with DNAse or 0.01-0.50 mol/L for incubation with RNAse, pH 6.0-8.5, additionally adding MgCl2 at 0.5-10.0 mmol/L, NaCl at 0.5-50.0 mmol/L and CaCl2 at 0.5-10.0 mmol/L, where the skin must remain under orbital agitation from 50 to 300 rpm for 30 min to 24 h, at a temperature of 20° C. to 40° C., with intervals for 2 to 7 solution changes;
b4) storing the skins in phosphate saline buffer solution or Tris-HCl or monobasic sodium phosphate/dibasic sodium phosphate or citrate/phosphate or 4-(2-hydroxyethyl)piperazine-1-ethanesulfonic acid at 0.025 -0.50 mol/L for incubation with DNAse or 0.01 -0.50 mol/L for incubation with RNAse, pH 6.0-8.5, with optional addition of MgCl2 to 0.5-10.0 mmol/L, NaCl to 0.5-50.0 mmol/L and CaCl2 to 0.5-10.0 mmol/L, with addition of DNAse or RNAse in concentration varying from 0.005 μg/mL to 0.5 g/mL, where the skin must remain under orbital agitation from 50 to 300 rpm for 30 min to 24 h, at a temperature from 20° C. to 40° C.;
b5) after the treatment with DNAse and/or RNAse, new exhaustive washing of the skin is carried out by the removal of the solution of (b4) and storing in said solution of (b2), where the skin must remain under orbital agitation from 50 to 300 rpm for 30 min to 24 h, at a temperature from 20° C. to 40° C., with intervals for 2 to 7 solution changes;
b6) for the incubation with protease, the exhaustive washing with the said solution in (b2) is replaced with the phosphate saline buffer solution or Tris-HCl or monobasic sodium phosphate/dibasic sodium phosphate or citrate/phosphate or 4-(2-hydroxyethyl)piperazine-1-ethanesulfonic acid at 0.025-0.50 mol/L, pH 6.0-8.5, with optional addition of MgCl2 at 0.5-10.0 mmol/L, NaCl at 0.5-50.0 mmol/L, CaCl2 at 0.5-10.0 mmol/L and ethylenediaminetetraacetic acid 1.0-5.0 mmol/L, with the protease(s) chosen in concentrations varying from 0.005 μg/mL to 0.5 μg/mL, where the skin must remain under orbital agitation from 50 to 300 rpm for 30 min to 24 h, at a temperature from 20° C. to 40° C.;
b7) after the treatment with protease, the exhaustive washing is carried out by the removal of the solution of (b6) and storing of the skin in the phosphate buffer saline solution or Tris-HCl or monobasic sodium phosphate/dibasic sodium phosphate or citrate/phosphate or 4-(2-hydroxyethyl)piperazine-1-ethanesulfonic acid at 0.025-0.50 mol/L, pH 6.0-8.5, with addition of CaCl2 100.0-200.0 mg/L and MgCl2 100.0-150.0 mg/L, where the skin must remain under orbital agitation from 50 to 300 rpm for 30 min to 24 h, at a temperature from 20° C. to 40° C., with intervals for 2 to 7 solution changes;
wherein the said protease in (b6) is selected from the group comprising: trypsin, subtilisin, collagenase, dispase, bromelain, pepsin or combinations thereof.

9. Process, according to claim 1, characterized in that step (c) comprises the sub-steps:
c1) incubation of the skin in sterile container containing bactericidal agent at 0.005-1.0% (m/v), for 15-60 minutes, under agitation from 50 to 300 rpm, at a temperature of 20° C. to 40° C., followed by rising in ultrapure sterile water in the same container for 15-60 minutes, in the same conditions of agitation and temperature, from one to ten repetitions; wherein the bactericidal agent is selected from the group that comprises: chlorhexidine digluconate, sodium chlorite, cetylpyridinium chloride, chloramine T, sodium dichloroisocyanurate, optionally the bactericidal agent is the chlorexidine digluconate;
c2) incubation of the skin in sterile container containing acetic acid/acetate buffer or glycine/HCl or citric acid/citrate or monobasic sodium phosphate/dibasic sodium phosphate at 0.025-0.50 mol/L, pH 3.0-6.0, for 30-120 minutes, under agitation from 50 to 300 rpm, at a temperature from 20° C. to 40° C., followed by incubation with ultrapure sterile water in the same container for 15-60 minutes, in the same conditions of agitation and temperature, in one to ten repetitions;
c3) incubation of the skin in sterile container containing Tris-HCl buffer or phosphate saline buffer or monobasic sodium phosphate/dibasic sodium phosphate or citrate/phosphate or 4-(2-hydroxyethyl)piperazine-1-ethanesulfonic acid (HEPES) at 0.025-0.50 mol/L, pH 6.0-8.5, for 30 minutes-24 hours, under agitation from 50 to 300 rpm, at a temperature from 20° C. a 40° C., in five to thirty repetitions.

10. Process, according to claim 3, characterized in that additional step (i) comprises:
incubation of the skin in sterile container containing Hanks solution or phosphate buffer saline solution or 4-(2-hydroxyethyl)piperazine-1-ethanesulfonic acid or 2-(N-morpholino)ethanesulfonic) acid (MES) at 0.015-0.50 mol/L, pH 3.0-8.5, for 30 to 360 minutes, under agitation from 50 to 300 rpm, at a temperature from 20° C. to 40° C., in one to five repetitions;
incubation of the skin in sterile container containing Hanks solution or phosphate buffer saline solution or 4-(2-hydroxyethyl)piperazine-1-ethanesulfonic acid (HEPES) or 2-(N-morpholino)ethanesulfonic) acid (MES) at 0.015-0.50 mol/L, pH 3.0-8.5, with addition of crosslinking reagent at 0.01%-2.0% or 0.01-50.0 mg/mL or 0.05-500 μg/mL for 30 minutes to 24 hours, under agitation from 50 to 300 rpm, at a temperature from 20° C. to 40° C., in one to five repetitions;
incubation of the skin in sterile container containing physiological solution for 30 minutes to 24 hours, under agitation from 50 to 300 rpm, at a temperature from 20° C. to 40° C., in one to fifteen repetitions.
wherein the crosslinking agents are selected from the group comprising: glutaraldehyde, genipin, N-hydroxysuccinamide, N-(3-dimethylaminepropyl)-N-ethylcarbodiimide, procyanidin, transglutaminase or combinations thereof.

11. Process, according to claim 3, characterized in that the additional step (ii) comprises:
incubated skin with a 10% hydrogen peroxide solution, under agitation from 50 to 300 rpm, temperature from 20° C. to 40° C., for a period from 30 min to 24 h;
exhaustive washing of the skin with ultrapure water;
incubation of the skins in saline phosphate buffer for a period from 30 min to 24 h;
exhaustive washing with sterile ultrapure water and incubation with physiological solution, under agitation from 50 to 300 rpm, temperature from 20° C. to 40° C., with intervals for solution change, for a period from 1 h to 24 h.

12. Process, according to claim 1, characterized in that step (d) comprises being in a freeze-dryer in a range from −30° C. to −80° C., with inner pressure lower than 50 μmHg, optionally in the range of 30 to 35 μmHg, and time from 2 to 24 h, followed by vacuum sealing of the skins in sterile plastic packaging with thickness from 0.15 to 0.40 pm; and by step (e) comprising the radiosterilization by gamma rays in Cobalt 60 radiator, with dosages that vary between 5 to 50 kGy.

13. Decellularized extracellular matrix, characterized by being obtained as defined in any one of claims 1 to 14.

14. Use of the decellularized extracellular matrix, as defined in claim 13, characterized by being for the production of medical, chemical, pharmaceutical, veterinary, dentistry products, to treat rupture of several tissues, dermatitises, acute, chronic and traumatic wounds, battlefield wounds, necrotic wounds, lacerations, abrasions, bruises, necrotizing fasciitis, epidermic necrolysis, Stevens Johnson syndrome, pressure wounds, ulcers due to venous insufficiency, arterial ulcers, diabetic or neuropathic ulcers, mixed ulcers, mucormycosis, vasculitis wounds, gangrenous pyoderma, reconstructions of abdominal wall for hernia repair, replacement of dura-mater, dural repair, correction of myelomeningocele and encephalocele, tympanoplasty, treatment of second and third degree burns, enterocutaneous graft, periodontal graft, inguinal hernia, rectovaginal fistula, anal fistula, eyelid reconstruction, nasal septum repair, nasosinusal repair, reconstruction of nasal and buccal lining, buccal mucous lesions, hiatal hernia, ventral hernia, rectal prolapse, Peyronie disease repair, urethra and ureter reconstruction, pelvic floor prolapse, pericardium repair, esophageal lesions due to trauma or tumor, reconstruction of cardiac valves, use in cardiovascular surgeries, congenital vaginal agenesis, neovagina construction, vaginal reconstruction, sexual reassignment in transgenders, breast prosthesis wrap, fat grafting pouch, genital prolapse, tympanic reconstruction, skin lesions and surgical reconstruction in animals, filling of oral mucosa, dental cavity and alveoli, as a mesh or suture material in the production of suture thread, or used to strengthen mesh or suture material; whereby the matrix can be used by itself or additionally it can be incorporated to primary, permanent, stem cells, associated to growth factors, recombinant proteins, drugs or natural products or combinations thereof.

15. Kit, characterized by comprising the decellularized extracellular matrix as defined in claim 13.
